(19) United States
(12) Patent Application Publication
(10) Pub. No.: US 2022/0328132 A1
(43) Pub. Date: Oct. 13, 2022

(54) NON-INVASIVE METHODS AND SYSTEMS FOR DETECTING INFLAMMATORY BOWEL DISEASE

(71) Applicant: Phyla Technologies Inc., Montreal (CA)

(72) Inventors: Ryszard Kubinski, Montreal (CA); Ryan Martin, Montreal (CA); Jean-Yves Ngassa Djamen-Kepaou, Montreal (CA); Timur Zhabanaev, Montreal (CA)

(73) Assignee: Phyla Technologies Inc., Montreal (CA)

(21) Appl. No.: 17/717,916

(22) Filed: Apr. 11, 2022

Related U.S. Application Data
(60) Provisional application No. 63/174,208, filed on Apr. 13, 2021.

(51) Int. Cl. G16B 25/10; G16B 40/20; G16H 10/40; G16H 50/20

(52) U.S. Cl. CPC G16B 25/10; G16B 40/20; G16H 10/40; G16H 50/20

(57) ABSTRACT

Methods, devices, and systems for detecting inflammatory bowel disease are described herein. The method includes obtaining a biological sample of a subject, determining sequencing data from the biological sample, and preprocessing the sequencing data. The preprocessing includes filtering the sequencing data to remove rare features, normalizing the filtered sequencing data to remove sequencing coverage variability, and batch effect reducing the filtered and normalized sequencing data. The method further includes calculating a likelihood of inflammatory bowel disease with a machine learning model using the preprocessed data as inputs.

### CROSS-REFERENCE TO PREVIOUS APPLICATION

This application claims priority from U.S. provisional patent application No. 63/174,208 filed on Apr. 13, 2021, which is incorporated herein by reference in its entirety.

### FIELD

The present disclosure generally relates to methods and systems for performing diagnosis. In particular, the disclosure relates to methods and systems for determining the likelihood of inflammatory bowel disease in a subject

### INTRODUCTION

The human gut microbiome is a collection of microbes, viruses and fungi that reside in the human digestive tract. The gut microbiota plays an important role in human health, influencing food digestion, the immune system, circadian rhythm, and numerous other functions.

Alterations in the gut microbiome have been linked to illnesses such as type two diabetes, cirrhosis, psoriasis, multiple sclerosis, and inflammatory bowel disease (IBD). IBD comprises two subtypes: Crohn's disease (CD) and ulcerative colitis (UC). CD and UC are characterized by periodic inflammation throughout the gastrointestinal tract or localized to the colon, respectively.

Despite limited understanding of the disease's etiology, the increasing prevalence of IBD globally between 1990 and 2017 has been linked to the Western diet. Currently, IBD diagnosis and monitoring is primarily performed via blood tests, endoscopies, and fecal calprotectin which can be costly, invasive, and display variable accuracy, all of which leads to delayed diagnosis and infrequent disease monitoring.

Therefore, there is a need for non-invasive, low-cost, and rapid methods and systems for detecting inflammatory bowel disease.

### SUMMARY

The following summary is intended to introduce the reader to the more detailed description that follows, and not to define or limit the claimed subject matter.

According to one aspect of the present disclosure, there is provided a method of determining the likelihood of inflammatory bowel disease (IBD) in a subject. The method comprises determining sequencing data from a biological sample of a subject. The method further comprises preprocessing the sequencing data. The preprocessing includes filtering the sequencing data to remove rare features, normalizing the filtered sequencing data to remove sequencing coverage variability, batch effect reducing the filtered and normalized sequencing data; and generating engineered features from the sequencing data. The method also comprises calculating a likelihood of inflammatory bowel disease with a machine learning model trained and tested using a preprocessed initial dataset, the preprocessed sequencing data being used as inputs to the machine learning model.

In some examples, the method further comprises obtaining a biological sample of a subject.

In some examples, generating engineered features from the sequencing data may further comprise generating any combination of the following engineered features: alpha diversity, types of bacterial interactions, a dysbiosis index, Firmicutes to Bacteroidetes ratio, gut microbiome health index (GMHI), healthy plane score, microbiome novelty score, principal component analysis score, and single sample network perturbation analysis score.

In some examples, the rare features comprise any one of one or more rare bacteria, and one or more features that do not appear in a pre-determined proportion in the initial dataset.

In some examples, the rare features comprise one or more features that are present in 10% or less of samples in the initial dataset.

In some examples, the normalizing step comprises any one of centered-log ratio (CLR) normalization, isometric log-ratio (ILR) normalization, total sum scaling (TSS) transformation, and arcsine square root transformation (ARS).

In some examples, the batch effect reducing step comprises any one of naive zero-centering, an empirical Bayes method and a negative binomial regression method.

In some examples, the sequencing data comprises 16S rRNA gene data.

In some examples, the method further comprises processing the sequencing data from the 16S rRNA gene into features comprising operational taxonomic units (OTUs), bacterial genera and/or bacterial species.

According to another aspect of the present disclosure, there is provided a method for training a machine learning model. The method comprises preprocessing an initial dataset of sequencing data to produce a preprocessed initial dataset, the initial dataset comprising non-overlapping first and second subsets. The preprocessing includes filtering the initial dataset to remove rare features, normalizing the filtered initial dataset to remove sequencing coverage variability, batch effect reducing the filtered and normalized initial dataset, and generating engineered features from the sequencing data. The method further comprises, in one iteration of training the machine learning model, training the machine learning model using the first subset of the preprocessed initial dataset. The method further comprises evaluating a performance of the trained machine learning model using the second subset of the preprocessed initial dataset. The method further comprises performing a further iteration of training the machine learning model based on whether a pre-determined level of performance is achieved.

In some examples, the step of generating engineered features from the sequencing data comprises generating any combination of the following engineered features: alpha diversity, types of bacterial interactions, a dysbiosis index, Firmicutes to Bacteroidetes ratio, gut microbiome health index (GMHI), healthy plane score, microbiome novelty score, principal component analysis score, and single sample network perturbation analysis score.

In some examples, the machine learning model comprises any one of: a random forest (RF) model, a k-nearest neighbors (KNN) model, a neural network, a logistic regression model, and a decision tree model.

In some examples, the rare features comprise any one of one or more rare bacteria, and one or more features that do not appear in a pre-determined proportion in the initial dataset.

In some examples, the rare features comprise one or more features that are present in 10% or less of samples in the initial dataset.

In some examples, the normalizing step comprises any one of centered-log ratio (CLR) normalization, isometric log-ratio (ILR) normalization, total sum scaling (TSS) transformation, and arcsine square root transformation (ARS).

In some examples, the batch effect reducing step comprises any one of naive zero-centering, an empirical Bayes method and a negative binomial regression method.

According to yet another aspect of the present disclosure, there is provided a system comprising one or more computers and one or more storage devices storing instructions that are operable, when executed by the one or more computers, to cause the one or more computers to perform operations the above method of determining the likelihood of inflammatory bowel disease (IBD) in a subject.

According to yet another aspect of the present disclosure, there is provided a system comprising one or more computers and one or more storage devices storing instructions that are operable, when executed by the one or more computers, to cause the one or more computers to perform operations of the above method for training a machine learning model.

### DRAWINGS

In order that the claimed subject matter may be more fully understood, reference will be made to the accompanying drawings, in which:

FIG. 1 is a block diagram of a non-invasive method for detecting inflammatory bowel disease in accordance with an embodiment of the present disclosure;

FIG. 2 is a block diagram of data preprocessing method for the method of FIG. 1;

FIG. 3 is a block diagram of a method for using a machine learning model model for predicting inflammatory bowel disease in accordance with an embodiment of the present disclosure;

FIG. 4 is a block diagram of a method for training a machine learning model for predicting inflammatory bowel disease in accordance with an embodiment of the present disclosure;

FIG. 5 is a block diagram of a method for evaluating a machine learning model for predicting inflammatory bowel disease in accordance with an embodiment;

FIG. 6 shows the results for a number of models in accordance with the disclosure;

FIG. 7 shows a number of datasets used for model generation; and

FIG. 8 shows a block diagram of a computer system implementing the methods for detecting inflammatory bowel disease described herein.

### DESCRIPTION OF VARIOUS EMBODIMENTS

Various systems and methods will be described below to provide an example of each claimed embodiment. No embodiment described below limits any claimed embodiment and any claimed embodiment may cover methods and systems that differ from those described below. The claimed embodiments are not limited to systems and methods having all of the features of any one system and method described below or to features common to multiple or all of the apparatuses described below.

One or more systems described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, and personal computer, cloud-based program or system, laptop, personal data assistance, cellular telephone, smartphone, or tablet device.

Each program is preferably implemented in a high-level procedural or object-oriented programming and/or scripting language to communicate with a computer system. However, the programs can be implemented in assembly or machine language, if desired. In any case, the language may be a compiled or interpreted language. Each such computer program is preferably stored on a storage media or a device readable by a general or special purpose programmable computer for configuring and operating the computer when the storage media or device is read by the computer to perform the procedures described herein.

A description of an embodiment with several components in communication with each other does not imply that all such components are required. On the contrary a variety of optional components are described to illustrate the wide variety of possible embodiments of the present invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

Over the past decade, several studies have investigated the different gut microbiome profiles of healthy individuals and those with CD or UC. Common characteristics of the gut microbiome identified in patients with IBD is a reduction in bacterial diversity and development of a dysbiotic state, referring to alterations in the structure and function of the gut microbiome compared to healthy individuals. Principal coordinate analysis with UniFrac or Bray-Curtis distance of the gut microbiome's composition have identified differential clustering of healthy and IBD samples. Although the dysbiotic state is commonly identified in IBD patients, it remains unknown whether the microbiome initiates IBD or is only a reflection of the patient's current health status. Previous studies with specific patient cohorts or larger meta-analyses have aimed to identify differentially abundant taxa between IBD patients and healthy controls in order to generate potential diagnostic biomarkers, although they have had limited success in the clinic to date.

The limited success of a PCR-based diagnostic test for microbiome biomarkers has led the field to apply predictive machine learning (ML) models to guide biomarker identification and/or improve classification of patient phenotypes. ML models are well suited to extract predictive information from the complex and multi-dimensional nature of microbiome data. In fact, several studies have demonstrated accurate classification of patients with IBD and whether a patient will achieve remission following pharmacological intervention based on their gut microbiome profile using ML models. Common ML models employed for IBD classification include Random Forest (collection of decision trees for classification), logistic regression (binary linear classifier) and neural networks (layers of differently weighted nodes contributing to a classification).

Features commonly used for IBD classification machine learning (ML) models can be categorized into two groups: bacterial and clinical. Clinical features encapsulate those regarding the patient (e.g., age, sex, BMI) and results from other clinical tests (e.g., calprotectin) that are separate from the microbiome profile. Bacterial features are determined from amplicon-based next generation sequencing (NGS) of the hypervariable regions in the bacterial 16S rRNA gene. Bioinformatic tools, such as QIIME2 or Mothur, cluster 16S rRNA-amplicon sequences into operational taxonomic units (OTUs) which are assigned to a specific taxonomy using large databases of bacterial rRNA genes, such as Silva or Greengenes.

One consideration for generating machine learning models for disease classification is their generalizability to previously unseen cohorts of patients. A model that misclassifies when presented with data from a new patient cohort cannot be applied in a clinical setting. Current models are often trained and tested using cross-validation with different splits of data from the same cohort. When cross-validation with an unseen sample cohort is performed, the performance of models is often lower, indicative of model overfitting to the training set. A proposed explanation for the reduced performance is the potential for non-biological variability introduced to the data by wet-lab protocols and sequencing instruments during the processing of these samples.

In order to improve generalizability, it is desirable to utilize the appropriate raw sequencing data preprocessing pipeline paired with the appropriate normalization and batch effect reduction techniques prior to model training and testing.

According to an embodiment of the present disclosure, there is provided a method for fecal microbiome-based classification of Inflammatory Bowel Disease (IBD) from a fecal sample. For example, the method includes receiving 16S rRNA gene sequencing data; preprocessing the sequencing data; using preprocessed data as features for machine learning classification of the IBD status of a sample. Thus, the present subject matter relates to techniques for profiling and classifying the microbiome for the purpose of IBD presence detection.

For example, the present subject matter teaches a method for aiding in disease diagnosis of IBD from a stool sample. Raw sequencing data from a stool sample is preprocessed and then used in pre-trained machine learning models in order to generate a prediction of IBD disease status. To elaborate, a stool sample intended for analysis by the present method can be collected using a collection kit, which is known in the art. Collected stools may be processed in the laboratory in order to extract and amplify the bacterial DNA. The 16S rRNA gene may then be sequenced. Raw sequencing data from the 16S rRNA gene may be processed via a bioinformatic pipeline into taxonomic features such as operational taxonomic units (OTUs), bacterial genera or bacterial species. These features may then be filtered and normalized and batch effects can be removed. These features may also be transformed by various feature engineering methods to generate disease scores or extract additional information about the microbiome.

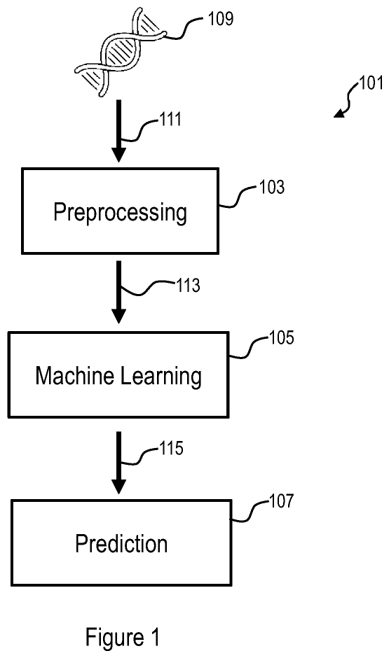

A stool sample's normalized, filtered and batch effect free taxonomic feature abundance vector and feature engineered values can then be used as input for a pre-trained machine learning model to generate a disease classification of IBD-positive or IBD-negative. As shown in FIG. 1, there is shown a block diagram of a non-invasive method 101 for detecting inflammatory bowel disease in accordance with an embodiment. Sequencing data 109, such as fecal microbiota 16S gene raw sequences, are provided to a preprocessing module 103, for example, through a data preprocessing pipeline 111. The preprocessing module 103 can be configured to preprocess the sequencing data. For example, the preprocessing module 103 can process the sequencing data 109 by filtering the data, normalizing the data, reducing batch effect in the data, and generating additional engineered features. Processing the sequencing data by filtering, normalizing, reducing batch effect, and feature engineering provides significant advantages to the methods and systems disclosed in the present subject matter.

One of the technical problems with available sequencing datasets is that they are collected and processed at different locations (for e.g., hospitals in different countries), with, for example, different sample collection methods, different storage conditions/sampling time points, different DNA extraction kits, etc. For example, two different hospitals can produce two different sequencing datasets for the same person.

For example, microbiome sequencing data of Person A collected and sequenced at a first lab can be different from microbiome sequencing data of the same Person A but collected and sequenced at a second lab. Therefore, to account for this discrepancy in data collection, storage conditions, sampling time points and DNA extraction kits, it is highly desirable to process each dataset using the method and system by filtering the dataset, normalizing the dataset and reducing batch effects in the dataset. This leads to a more accurate machine learning model and a highly accurate prediction of IBD status.

This filtering step is distinct from sequence filtering that takes place at the very start of data processing. Sequence filtering for chimera removal is done using, for example, the Quantitative Insights Into Microbial Ecology (QIIME™) bioinformatics pipeline Accurate, High-Resolution Sample Inference from Amplicon Sequencing Data (DADA2) package.

Filtering the data removes outliers that could have a negative impact on the machine learning model's performance. Outliers exist due to issues with the laboratory methods used to generate the data as well as due to the presence of rare bacteria. For example, feature filtering can be performed to remove rare bacteria. For feature filtering, bacteria/features that are not commonly seen in the dataset are removed. For example, as microbiome datasets are small, the actual level of rare bacteria that should be removed cannot be precisely known, and there can be very little data points with these bacteria. To account for this, bacteria/features that do not appear in a significant proportion of the data points are removed. For example, for the filtering, features that are present in at least 10% of samples in at least one data source are retained and all other features are removed. In addition, feature filtering can include retaining bacteria/features that are identified at different abundance in samples from patients with IBD and those without. Following statistical tests, such as in the R package (a software environment freely provided by the R Foundation) Analysis of Compositions of Microbiomes with Bias Correction (ANCOM-BC), bacteria with differential abundance above a significance threshold (e.g., p>0.05) can be removed. The filtering step can be performed by a software script (or a software robot) that is programmed to execute the filtering step on the dataset. For example, the software script can be executed by module 103 (or the computer system) as disclosed herein.

By normalizing the data, the module 103 prepares the data for machine learning processing. For example, during the normalization process, module 103 can change some of the values in the dataset to use a common scale, without distorting differences in the ranges of values or losing information. For example, the normalization step is highly desirable as it allows the data to appear more uniform, allowing to produce a more accurate machine learning model. Normalization removes noise from data. This is especially important for microbiome data because each datapoint is sequenced to a slightly different depth, meaning that the absolute feature counts within the raw data are not comparable between data points unless the data is normalized.

Normalization methods can be implemented, for example, using a custom package or Python® and R packages with the methods already incorporated. For centered-log ratio (CLR) and isometric log-ratio (ILR), zero values are first replaced with the multiplicative replacement function prior to normalization with the CLR and ILR functions, respectively, from the Python® package sciKit-bio™, for example. CLR performs a log transformation of abundance values, which are normalized by the geometric mean of all features. ILR uses a change of coordinate space projection calculation to transform proportional data (or relative abundances) to a new space with an orthonormal basis. For total sum scaling (TSS), the counts for each feature can be divided by the sum of all feature counts in the sample with a custom python function, for example.

The normalization method can constrain the sample row sum to one, aiming to similarly scale all samples while maintaining biological information of microbial abundances. For arcsine square root transformation (ARS), the TSS normalized values can be transformed with sqrt function followed by the arcsin function from the Python® package NumPy™, for example. The log transformation (LOG) can also be applied to the TSS normalized values using the log function from NumPy™ following replacement of all 0s with 1s. The variance-Stabilizing-Transformation (VST) function in the R package Differential Gene Expression Analysis Based on the Negative Binomial Distribution (DESeq2) can be used, for example. VST aims to factor out the dependence of the variance in the mean abundance of a feature. The method can numerically integrate the dispersion relation of the feature mean fitted with a spline, evaluating the transformation for each abundance in the feature. The normalization step can be performed by a software script (or a software robot) that is programmed to execute the normalization step on the filtered dataset. For example, the software script can be executed by module 103 (or the computer system) as disclosed herein.

Module 103 is also adapted to reduce batch effects in the data. Batch effects can be sources of variation, such as different processing times or different handlers. For example, three methods for batch reduction can be used, naive zero-centering, an empirical Bayes method, and a negative binomial regression method. The naive method implemented can be zero-centered batch reduction, which entails centering the mean of each feature within each batch to zero.

For example, an empirical Bayes method can be implemented. The Bayes method can be designed specifically for zero-inflated microbial abundance data using the R package Meta-analysis Methods with a Uniform Pipeline for Heterogeneity (MMUPHin). MMUPHin estimates parameters for the additive and multiplicative batch effects, using normal and inverse gamma distributions, respectively. The estimated parameters can then be used to remove the batch effects from the dataset. In addition, the negative binomial regression method ComBat-seq fits the feature counts to a negative binomial regression model to estimate the batch specific parameters. The batch specific parameters are used to calculate a ‘batch-free’ distribution which the raw counts are mapped to in order to obtain the final corrected data. For MMUPHin and ComBat-seq, the sample type (stool/biopsy) can be used as a covariate for a first method and the sample type and disease label (UC/CD/Control) can be covariates for a second method.

A batch can be considered for the whole study or a study can be split into multiple batches when the metadata indicated different sample laboratory preprocessing methods. The batch effect reduction step can be performed by a software script (or a software robot) that is programmed to execute the batch effect reduction steps on the normalized and filtered dataset. For example, the software script can be executed by module 103 (or the computer system) as disclosed in the present application.

Feature engineering is the process of applying domain knowledge to extract additional information from a bacteria abundance vector. The engineered features are used as input to the machine learning model alongside the bacteria/feature abundance. In various non-limiting embodiments, any combinations of the following engineered features can be used in accordance with the presently disclosed systems and methods: alpha diversity, types of bacterial interactions, a dysbiosis index, Firmicutes to Bacteroidetes ratio, gut microbiome health index (GMHI), healthy plane score, microbiome novelty score, principal component analysis score, and single sample network perturbation analysis score.

Alpha diversity is a measure of the diversity of bacteria identified in the stool sample. Several different alpha diversity metrics can be implemented, including Shannon diversity, Simpson's index, Gini index, and Faith's phylogenetic diversity (Faith's PD). For example, Shannon diversity measures the evenness of abundance across the bacteria identified in the stool sample. The Simpson index provides the probability that the n-th bacteria drawn from the stool sample bacterial population will be the same genus as the (n-th)−1 bacteria. The Gini index measures the inequality in abundance of each bacterium in a stool sample, with an index of 0 indicating all bacteria have the same abundance. Faith's PD measures diversity by determining the sum of the branch lengths in a phylogenetic tree containing all bacteria within a stool sample.

Bacteria in the microbiome interact with each other to inhibit each other's growth (competitive), to improve one's growth and inhibit the other's growth (antagonistic), or to increase the growth of each bacterium (mutualistic). The types of interactions between the bacteria in a stool sample can be determined by flux balanced analysis (FBA) solved with linear and quadratic optimization programs, as implemented in software including but not limited to MICOM.

The dysbiosis index is the log ratio of the total abundance of bacteria increased and the total abundance of bacteria decreased in stool samples from unhealthy individuals compared to healthy individuals. The index can be determined for any disease of interest, including but not limited to, IBD.

The firmicutes to Bacteroidetes ratio compares the total abundance of bacteria within the Firmicutes phylum and the total abundance of bacteria within the Bacteroidetes phylum.

The gut microbiome health index (GMHI) measures the ratio of the total abundance of bacteria prevalent in some dataset of healthy stools samples compared to the total abundance of bacteria prevalent in another dataset of unhealthy stool samples. Unhealthy stool samples consist of those collected from individuals diagnosed with, but not limited to obesity, IBD, colorectal cancer, type II diabetes, rheumatoid arthritis, depression, and multiple sclerosis.

For the healthy plane score, bacteria/features identified in the stool sample are transformed to a 3-dimensional vector representation with a dimensionality reduction method, including but not limited to, principal component analysis or principal coordinate analysis. A 2-dimensional plane is fit within the 3-dimensional space by minimizing the Euclidian distance of some dataset of healthy stool samples to the 2-dimensional plane. The healthy plane score is determined as the Euclidian distance of a transformed 3-dimensional representation of the stool sample to the 2-dimensional plane.

Microbiome novelty score measures the similarity of the bacteria/features in a stool sample to a reference dataset of healthy samples. The similarity between stool samples is determined by, but not limited to, the beta diversity metrics Bray-Curtis distance and Weighted UniFrac distance. The score comprises the weighted average of the similarity between a stool sample and reference healthy samples, with those more similar given a higher weight.

For the method involving a principal component analysis score, dimensionality reduction of the bacteria/features within a batch of stool samples is performed by principal component analysis to reduce the sample to the top principal components. The top principal components across different batches are clustered by similarity of the bacteria/feature contributions to the principal component. The average bacteria/feature contribution within each cluster is determined and used to generate a cluster score for each stool sample from their bacteria/feature abundance vectors.

Single sample network perturbation analysis identifies how the relationship between bacteria in a stool sample is altered from a reference relationship network. The reference network, a directed acyclic graph, is learned from a collection of reference healthy stool samples. For each node (bacteria) in the network, the abundance is predicted from the abundance of the nodes (bacteria) in its Markov blanket with a regression machine learning model. For a single stool sample, the perturbation is determined by the prediction residuals, which is the difference in the predicted abundance and the bacteria's actual abundance. The perturbation score is therefore a vector of residuals for each bacterium in the stool sample.

At 113, the batch reduced, normalized, filtered and engineered features are then fed into a machine learning module 105. The machine learning module 105 is configured to apply a pre-trained machine learning model to the features to generate a classification value at 115 of IBD status for the sample. The classification 115 can then be used to generate IBD prediction 107.

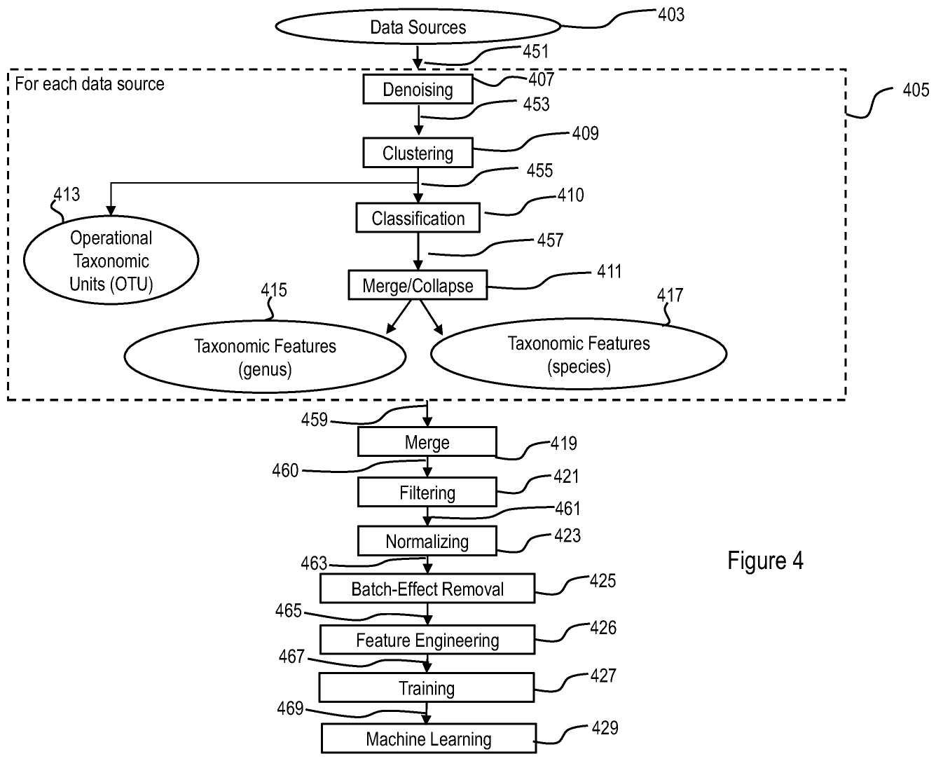

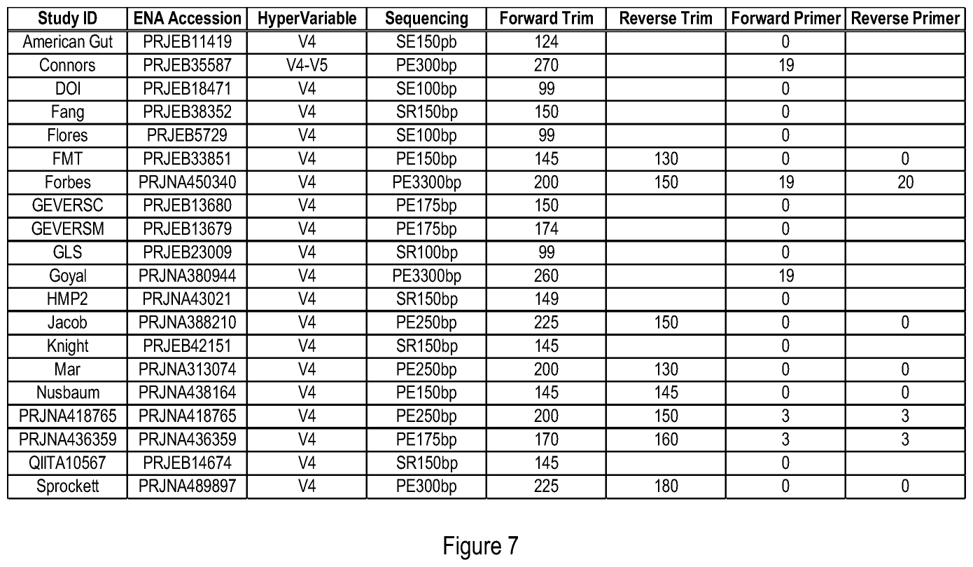

The machine learning models are pre-trained (see FIG. 4) on taxonomic features and clinical metadata that are generated from a collection of fifteen publicly available datasets of 16S rRNA gene raw sequencing data (see FIG. 7). As will be appreciated, other datasets could be used. The raw data is preprocessed separately and then merged into a collective singular dataset. From this collective dataset, the taxonomic features are filtered, normalized and have batch effects removed. The taxonomic features are also used to generate additional engineered features.

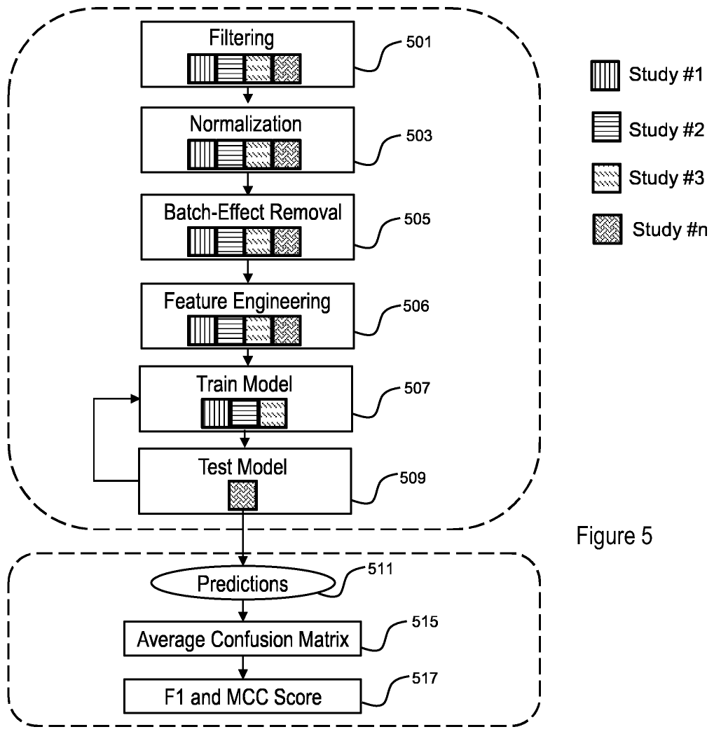

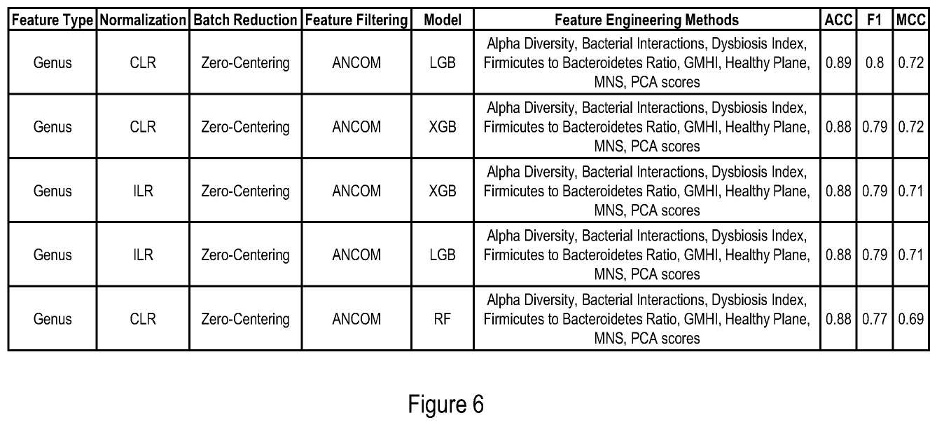

The performance of the different combinations of ML model and data preprocessing approach can be evaluated using a leave one dataset out approach (see FIG. 5), where model performance is averaged over n iterations where each iteration involves n−1 studies used for training and a different nth study used as a test set. Combinations that perform well in this validation indicate a high level of generalizability of the combination and therefore potential clinical applicability. The top performing combinations of data preprocessing and machine learning model are outlined in FIG. 6.

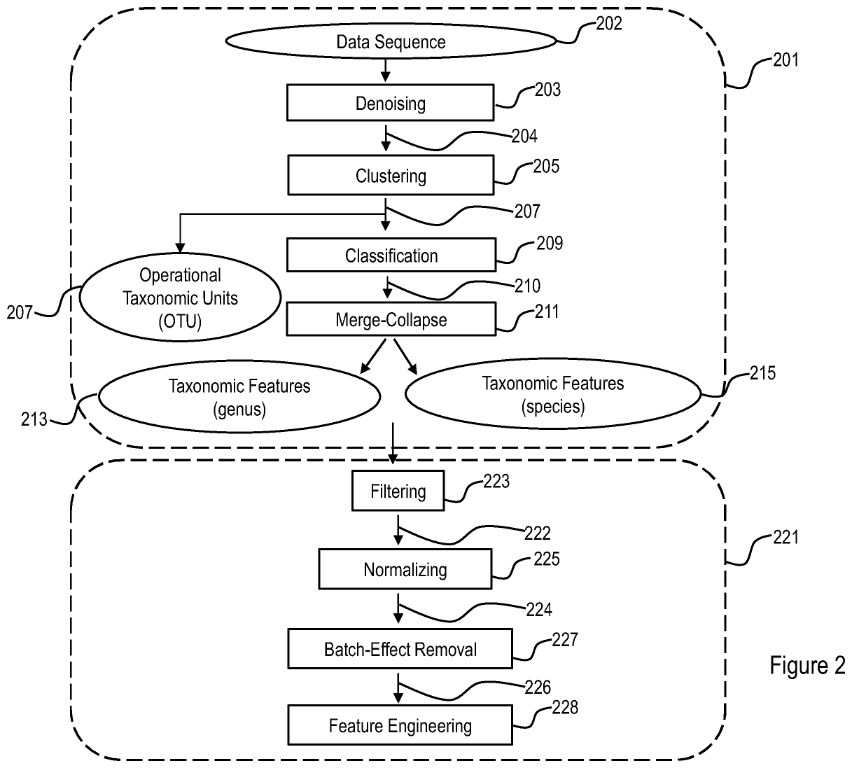

Referring to FIG. 2, there is shown, at 201, processing of data sequences into taxonomic features and there is shown, at 221, processing of taxonomic features via filtering, normalization and batch effect removal methods in order to prepare the features for input into a machine learning model.

During the processing steps 201, data sequences 202, such as 16S rRNA Gene Raw Sequences, are fed to a denoising module 203. The denoising module 203 can be configured to filter and remove low quality reads and/or chimeras in the data sequences 202. Trimming parameters, such as those disclosed in FIG. 7, can be used by the denoising module in order to trim the DNA sequences. The denoising module 203 can be configured to denoise the data sequences 202, for example, by using DADA2 or Deblur packages. Outputs 204 of the denoising module 203 can include filtered, trimmed and/or dereplicated to amplicon sequence variants. The outputs 204 are fed into a clustering module 205.

The clustering module can be adapted to apply to the output 204 a closed-reference OTU clustering at 99% identity against Silva 132 99% OTU database using Vsearch. For example, the clustering module 205 can be configured to cluster the sequences into Operational Taxonomic Units (OTU) 207 and the centroid sequences can then be classified with a Naive Bayes classifier at 99% confidence using the Silva 132 99% reference database. The OTUs from 207 can be fed into a taxonomic classification module 209. The classification module 209 can perform taxonomic classification on the OTU using Naive Bayes classifiers trained on Silva 132 database for 16S rRNA gene v3-v4 regions used at 99% confidence threshold.

The extracted reads and the corresponding taxonomy can be used to train the Bayes classifier with the QIIME2 plugin feature-classifier's fit-classifier-naive-bayes function. The trained classification module 209 can output bacterial taxonomies 210. The bacterial taxonomies can be fed into a merging/collapsing module 211. The merge-collapse module 211 can be configured to merge and collapse the taxonomies to a desired taxonomic level 213 and 215. For example, the taxonomy tables can be collapsed to level 6 (genus) at 213, level 7 (species) at 215, or simply used at the OTU level 207.

At 221, the taxonomic features 207, 213 and 215 can be filtered, normalized and processed to remove batch effects in order to prepare them for input into a machine learning model. The filtering module 223 can be configured to receive any one of the features 207, 213 and 215. For example, the filtering module 223 can be used to filter features that are used for model training. The features can be filtered using the following process. Features present in less than 10% of the samples within each study or those not differentially abundant between healthy and IBD samples can be pruned (for e.g., removed) from the dataset. The feature pruning can be performed on the training set only. The test set's features can be pruned to match those of the training set. Sample points with less than 4000 feature counts can be removed from the training and test sets.

After filtering, the filtered feature tables 222 can be normalized by the normalizing module 225. In some embodiments, to normalize the features, the normalizing module can use one of the following methods: centered-log ratio (CLR), isometric log-ratio (ILR), total sum scaling (TSS) or arcsine square root transformation (ARS).

In the centered-log ratio (CLR) and isometric log-ratio (ILR) methods, zero values are first replaced with the multiplicative replacement function prior to normalization with the CLR and ILR functions, respectively, from the python package SciKit-Bio (v0.5.2). In the total sum scaling (TSS) method, the counts for each feature are divided by the sum of all feature counts in the sample with a custom Python function. The method constrains the sample row sum to one, aiming to similarly scale all samples while maintaining biological information of microbial abundances. For arcsine square root transformation (ARS), TSS normalized values are transformed with sqrt function followed by the arcsin function from the Python® package NumPy™. The log transformation (LOG) can also be applied to the TSS normalized values using the log function from Numpy following replacement of all 0s with 1.

After normalization, normalized features 224 can have batch effects removed from them by the batch-effect removal module 227, for e.g., by using zero-centered batch reduction (BRZC). This can entail subtracting the mean of all values across all samples from all values in a study (setting the group mean to zero, i.e., one-way ANOVA). Following the process 221, the batch-effect removal module 227 can output the batch-effect-free, filtered and normalized features 226. In some embodiments, at step 228, feature engineering methods, as described in more detail elsewhere herein, can be applied to the batch-effect-free, filtered and normalized features.

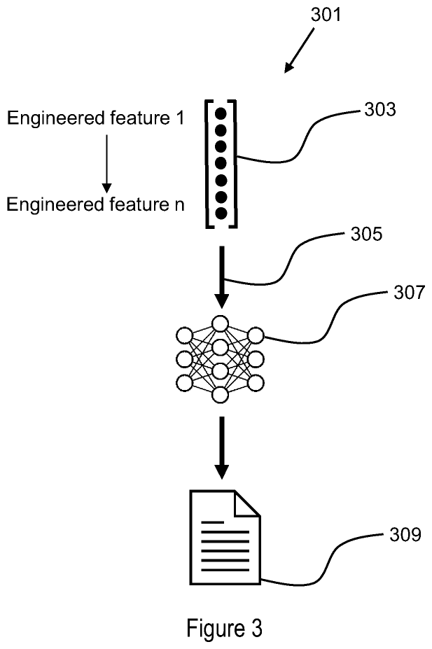

Referring to FIG. 3, there is shown a method 301 for detecting inflammatory bowel disease. At 305, a filtered, normalized and batch-reduced bacterial feature vector 303 is provided to a pre-trained machine learning module 307 such as, for example, Random Forest (RF) and XGBoost (XGB) or K-Neatest-Neighbours models. By using a pre-trained machine learning model, the module 307 is configured to generate an IBD prediction report 309 based on the feature vector 303. The IBD prediction report 309 contains a predictive value generated by the model.

Referring to FIG. 4, there is shown a method for training a machine learning model for predicting IBD. Data sources 403, such as sample FASTQ files, can be acquired from the European Nucleotide Archive (ENA) browser. The sample metadata can be acquired from the corresponding publication's supplementary materials or the QIITA microbiome platform. According to one example, only samples collected from individuals in North America are used from each study.

In some embodiments, the following twenty studies (see FIG. 7) are included in the dataset:
1. The American Gut cohort is from a large, open platform which collected samples from individuals in the US to identify associations between microbiomes and the environment and individual's phenotype (See Bryrup T, Thomsen C W, Kern T, et al., Metformin-induced changes of the gut microbiota in healthy young men: results of a non-blinded, one-armed intervention study, Diabetologia 2019; 62:1024-35). Available samples that did not contain any self-reported diseases in the metadata were included;
2. The Connors study assessed the relationship between bile acids and the fecal gut microbiome in a cohort of pediatric Crohn's disease patients administered exclusive enteral nutrition. (See Connors J, Dunn K A, Allott J, et al. The relationship between fecal bile acids and microbiome community structure in pediatric Crohn's disease. ISME J. 2020; 14: 702-713);
3. The DOI study assessed the variation in the gut microbiome over time and how the variation differed between patients diagnosed with IBD and healthy controls. (See Halfvarson J, Brislawn C J, Lamendella R, et al. Dynamics of the human gut microbiome in inflammatory bowel disease. Nat Microbiol. 2017; 2: 17004);
4. The Fang study assessed how different surgical interventions for IBD influenced the composition of the gut microbiome. 10.1093/ibd/izaa262;
5. The Flores study was a longitudinal study to assess variation temporal variation in the gut microbiome. (See Fang X, Vazquez-Baeza Y, Elijah E, et al. Gastrointestinal Surgery for Inflammatory Bowel Disease Persistently Lowers Microbiome and Metabolome Diversity. Inflamm Bowel Dis. 2021; 27: 603-616.);
6. The FMT study assessed the efficacy of fecal microbiome transplants to induce remission in patients with ulcerative colitis. (See de Leeuw M A, Duval M X. Selecting donors for fecal microbiota transplantation in ulcerative colitis. bioRxiv. medRxiv; 2020. Doi:10.1101/2020.03.25.20043182);
7. The Forbes study assessed the difference in composition of the gut microbiome in patients with CD, UC, multiple sclerosis, and rheumatoid arthritis in comparison to healthy controls. (See Forbes J D, Chen C-Y, Knox N C, et al. A comparative study of the gut microbiota in immune-mediated inflammatory diseases-does a common dysbiosis exist? Microbiome. 2018; 6: 221.);
8. The GEVERSC cohort consists of additional samples from pediatric and adult patients added to the GEVERSM study (See Gevers D, Kugathasan S, Denson L A, et al. The treatment-naive microbiome in new-onset Crohn's disease. Cell Host Microbe 2014; 15:382-92);
9. The GEVERSM study assessed the microbiome composition of treatment naive, newly diagnosed, pediatric patients with IBD and adult patients diagnosed with IBD for 0 to 57 years (See Gevers D, Kugathasan S, Denson L A, et al. The treatment-naive microbiome in new-onset Crohn's disease. Cell Host Microbe 2014; 15:382-92);
10. The GLS study longitudinally sampled 19 patients with CD (Crohn's disease activity index (CDAI) between 44 and 273) and 12 healthy control individuals (See Ma C, Battat R, Parker C E, et al. Update on C-reactive protein and fecal calprotectin: are they accurate measures of disease activity in Crohn's disease? Expert Rev Gastroenterol Hepatol 2019; 13:319-30);
11. The Goyal study assessed the efficacy of a fecal microbiome transplant in pediatric patients with CD, UC, indeterminate colitis (IC) (See Goyal A, Yeh A, Bush B R, et al. Safety, Clinical Response, and Microbiome Findings Following Fecal Microbiota Transplant in Children With Inflammatory Bowel Disease. Inflamm Bowel Dis. 2018; 24: 410-421);
12. The Human Microbiome Project (HMP2) study longitudinal tracked pediatric and adult patients ranging from newly diagnosed to diagnosed for 39 years. Diagnosis was confirmed by colonoscopy prior to enrollment in the study along with several other inclusion criteria listed in the corresponding publication (See E Penna F G C, Rosa R M, da Cunha P F S, et al. Faecal calprotectin is the biomarker that best distinguishes remission from different degrees of endoscopic activity in Crohn's disease. BMC Gastroenterol 2020; 20:35);
13. The Jacob study assessed the efficacy of a fecal microbiome transplant in patients with active UC to induce a state of remission. (See Jacob V, Crawford C, Cohen-Mekelburg S, et al. Single Delivery of High-Diversity Fecal Microbiota Preparation by Colonoscopy Is Safe and Effective in Increasing Microbial Diversity in Active Ulcerative Colitis. Inflamm Bowel Dis. 2017; 23: 903-911);
14. The Knight study completed a multi-omics analysis to understand how the gut microbiome is altered in patients with UC (See Mills R H, Dulai P S, Vazquez-Baeza Y, et al. Multi-omics analyses of the ulcerative colitis gut microbiome link Bacteroides vulgatus proteases with disease severity. Nat Microbiol. 2022; 7: 262-276.);
15. The Mar study assessed the difference in gut microbiome composition between ethnically distinct cohorts of UC patients. (See Mar J S, LaMere B J, Lin D L, et al. Disease Severity and Immune Activity Relate to Distinct Interkingdom Gut Microbiome States in Ethnically Distinct Ulcerative Colitis Patients. MBio. 2016; 7. Doi:10.1128/mBio.01072-16);
16. The Nusbaum study assessed the changes in composition of the gut microbiome following fecal microbiome transplant in pediatric patients with ulcerative colitis. (See Nusbaum D J, Sun F, Ren J, et al. Gut microbial and metabolomic profiles after fecal microbiota transplantation in pediatric ulcerative colitis patients. FEMS Microbiol Ecol. 2018; 94. doi:10.1093/femsec/fiyl33);
17. PRJNA418765 was a longitudinal study of patients with CD that were refractory to anti-TNF initiating ustekinumab assessed at week 0, 4, 6 and 22. To be included, patients required at least three months Crohn's disease history and a CDAI between 220 and 450 (See Hill-Burns E M, Debelius J W, Morton J T, et al. Parkinson's disease and Parkinson's disease medications have distinct signatures of the gut microbiome. Mov Disord 2017; 32:739-49);
18. PRJNA436359 was a longitudinal study of new onset and treatment naive pediatric patients with UC receiving a variety of medications at week 0, 4, 12, and 52. Inclusion criteria consisted of presence of disease beyond the rectum, Pediatric Ulcerative Colitis Activity Index (PUCAI) of 10 or more, and no previous therapy (See 74 Nguyen V Q, Jiang D, Hoffman S N, et al. Impact of Diagnostic Delay and Associated Factors on Clinical Outcomes in a U.S. Inflammatory Bowel Disease Cohort. Inflamm Bowel Dis 2017; 23:1825-31);
19. QIITA10567 samples consist of the control individuals in a study linking alterations in microbiome composition to Parkinson's disease (See Vadstrup K, Alulis S, Borsi A, et al. Cost Burden of Crohn's Disease and Ulcerative Colitis in the 10-Year Period Before Diagnosis-A Danish Register-Based Study From 2003-2015. Inflamm Bowel Dis 2020; 26:1377-82); and
20. The Sprockett study assessed the effect of antibiotics on the composition of the gut microbiome in pediatric patients with CD. (See Sprockett D, Fischer N, Boneh R S, et al. Treatment-Specific Composition of the Gut Microbiota Is Associated With Disease Remission in a Pediatric Crohn's Disease Cohort. Inflamm Bowel Dis. 2019; 25: 1927-1938).

As will be appreciated by the skilled reader, other combinations of datasets could be used with the methods and systems disclosed herein.

At 405, there is disclosed data processing steps for training the machine learning model. At 405, each data source can be processed separately. The data can be processed as outlined in the data processing section 201 in FIG. 2. Taxonomy feature tables from each study can be collapsed to species (level 7) and genus (level 6) and then merged (for the same feature type) for further analysis. For example, this can result in 3 taxonomy tables for 15 studies: 1 with species features, 1 with genus features, 1 with OTU features.

Referring back to FIG. 4, at 451, each data source 403 is fed to a denoising module 407. The denoising module 407 can be configured to filter and remove low quality reads and/or chimeras in the data source 403. For example, trimming parameters, such as those disclosed in FIG. 7, can be used by the denoising module. The denoising module 407 can also be configured to denoise the data 403, for example, by using DADA2. Outputs data 453 of the denoising module 407 can include filtered, trimmed and/or dereplicated amplicon sequence variants. The outputs 453 are fed into a clustering module 409.

The clustering module can be adapted to apply to the output 453 a closed-reference OTU clustering at 99% identity against Silva 132 99% OTU database using a searching tool such as Vsearch or similar. For example, the clustering module 409 can be configured to cluster the output 453 into Operational Taxonomic Units (OTU) 413 and the centroid sequences classified with a Naive Bayes classifier at 99% confidence using the Silva 132 99% reference database. At 455, the OTU 413 can be fed into a classification module 410. The classification module 410 can perform taxonomic classification on the OTU using Naïve Bayes classifiers trained on Silva 132 database for 16S rRNA gene v3-v4 regions used at 99% identity threshold.

The extracted reads and the corresponding taxonomy can be used to train a Naive Bayes classifier with the QIIME2™ plugin feature-classifier's fit-classifier-naive-bayes function, for example. The classification module 410 can output bacterial taxonomies 457. The bacterial taxonomies can be fed into a merging/collapsing module 411. The merge-collapse module 411 can be configured to merge and collapse the taxonomies to a desired taxonomic level 415 and 417. For example, the taxonomy tables can be collapsed to level 6 (genus) at 415, level 7 (species) at 417, or simply used at the OTU level 413.

At 459, taxonomy features are fed to a merging module 419. The merging module 419 can be configured to merge datasets for the same feature types from different sources (for e.g., merge genus data from study 1 with all genus data from study 2 to study n, etc.). At 460, merged datasets from the merging module 419 are fed into a filtering module 421. In some embodiments, the filtering module 421 can be configured to filter data points with less than 4000 features counts form the merged datasets. In some embodiments, the filtering module 421 can further be configured to retain only features that appear in over 10% (for example) of data points in the merged datasets.

At 461, the filtered features output by the filtering module 421 are fed into a normalizing module 423. The normalizing module 423 can be configured to normalize feature counts using a normalizing method such as isometric log ratio (ILR), centered log ratio (CLR), arc sin root (ARS), and total sum scaling (TSS). At 463, the normalized features output by the normalizing module 423 are fed into a batch-effect removing module 425. The batch-effect removing module 425 can be configured to remove batch effects from the features data using for e.g., a zero-centering approach (BRZC).

At 465, the batch-effect-free, filtered and normalized features are provided to a feature engineering module 426 that applies feature engineering methods as described in more detail elsewhere herein.

At 467, the batch-effect-free, filtered, normalized and engineered features are provided to a training module 427. The training module 427 can be configured to train and test different machine learning modules using a leave-one-dataset-out approach to determine the best combination of data preprocessing and machine learning model. At 469, the training module 427 outputs a trained and tested machine learning model 429.

Referring to FIG. 5, there is shown a leave-one-study-out approach for evaluating model performance across a study collection including Study 1, Study 2, Study 3 and Study n.

As shown in FIG. 5, after filtering a step 501, the normalizing module 503 is configured to normalize each study dataset in the study collection including datasets for Study 1, Study 2, Study 3 and Study n. For example, the normalizing module can be configured to normalize feature counts in each dataset using a normalizing method as previously-described such as isometric log ratio (ILR), centered log ratio (CLR), arc sin root (ARS), and total sum scaling (TSS). The normalized datasets are feds into a batch-effect removing module 505, which can be configured to remove batch effects from the data using for e.g., a zero-centering approach. Then, at step 506, feature engineering is performed, as described in more detail elsewhere herein. Afterwards, the machine learning model is trained at the training module 507 using a leave-one-dataset-out approach. As shown in FIG. 5, the training module 507 can be configured to train the machine learning model by using the datasets (for e.g., batch-effect free, normalized and filtered datasets) for Study 1, Study 2 and Study 3. In this particular example, the data from Study n is not used for training of the machine learning model. The machine learning model trained by the training module 507 is inputted into the testing module 509. The testing module 509 can be configured to test the machine learning model using the data from Study n, for e.g., by comparing disease state predictions 511 of the machine learning model to clinical metadata from Study n.

At 513, a performance metric can be calculated with predictions from all interactions. At step 515, the normalized confusion matrix for each study can be generated. At step 517, different metrics, such as F1 score, binary accuracy, AUC and MCC, can be calculated for the trained machine learning models based on the predictions 511 and average normalized confusion matrix from all studies. Metrics for different machine learning models are shown in FIG. 6.

During model training and selection, classification can be implemented by machine learning models, such as Random Forest, XGBoost (XGB), light Gradient Boosting Machine (LGB). The models can be implemented using the python package SciKit-Learn, for example. For example, Random Forest (RF) models can use an ensemble of decision trees that discriminate the feature space by a sequence of greater or less than statements. The power of the model comes from its non-linear classification capabilities and the sheer number of trees used to label classification. The Random Forest classifier can be implemented with the following modifications to the default SciKit-learn settings: n_estimators=500, max features=sqrt, and class_weight=balanced. In addition, these parameters can be optimized through grid search or Bayesian hyperparameter optimization methods.

Referring to FIG. 6, to measure the performance of the various normalization, batch reduction and model combinations, four commonly used metrics for binary classification can be used: F1 score, area under the Receiver operating characteristic curve (AUC), binary accuracy and Matthews Correlation Coefficient (MCC). As the number of samples in a study ranged from 23 to 1,279, there was potential for the overall performance metrics to be skewed by the predictions of a single study with a large number of samples. Whereas in a typical 5-fold cross-validation each fold is weighted equally, with the classification performance determined separately for each fold and then an overall average calculated. Therefore, in order to calculate the pipeline's classification performance with equal weighting to each study, the confusion matrix for each study was generated and normalized by the number of samples in the study. The average proportion of true positives, true negatives, false positives, and false negatives across the 15 studies was used to generate an overall confusion matrix. The overall F1 score and MCC were calculated from the averaged confusion matrix with the following equations.

The generalizability of each model, normalization and batch reduction method can be determined through implementation of a cross validation strategy which may assess predictive performance on previously unseen batches of samples. As there are 15 datasets in some embodiments, the method can iterate through the dataset 15 times, generating the training set by removing all samples from a single study to a separate test set. Results of some of the performing combinations that are selected for the present subject matter are listed in FIG. 6.

Laboratory Procedure

A given stool sample may be collected through a collection method. Preferably, a cell lysing solution paired with a DNA stabilization buffer similar to that used by the DNA Genotek OMNI-200 kit is used. DNA can then be extracted using a validated DNA extraction method, such as the protocol and instruments used in the Qiagen PowerFecal DNA extraction kit. Extracted DNA can then have its 16S rRNA gene V4 region amplified using 515F-806R primers as specified in the Earth Microbiome Project. The DNA can then be barcoded and sequenced on an Illumina sequencing machine such as the iSeq or MiSeq that is capable of doing paired end, 250 bp sequencing. Raw sequencing data can be demultiplexed and assigned to its appropriate sample's FASTQ file.

Raw sequencing data can then be processed into taxonomic features as outlined in the present subject matter. For a given stool sample, the use of genus level data is proposed, paired with ILR normalization, and zero-centered batch reduction prior to prediction generation using a Random Forest model. Predictive values generated by the Random Forest model can then be used as a diagnostic aid.

Computer System

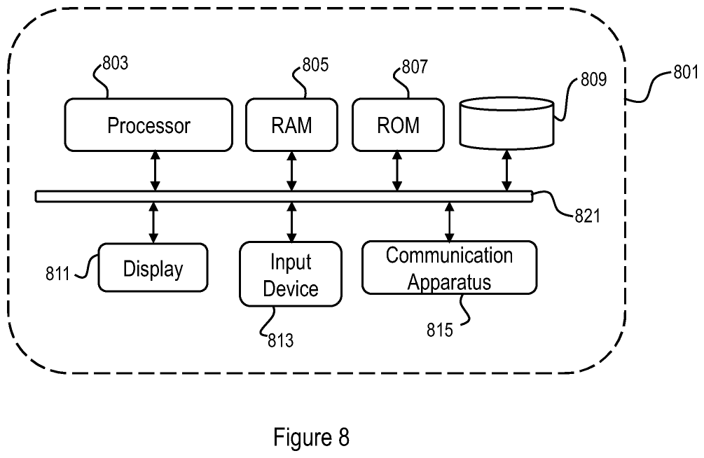

Referring to FIG. 8, there is a block diagram that illustrates a computer system 801 upon which, embodiments or portions of the embodiments, of the present disclosure may be implemented. The methods and modules described in the present subject matter can be implemented using the computer system 801 as a standalone device or on a distributed network of shared computer processing resources such as a cloud computing network. For example, a non-transitory computer-readable medium can be provided in which a computer program is stored for causing a computer to perform the disclosed methods for detecting inflammatory bowel disease. The non-transitory computer-readable medium can be provided in which the computer program is stored for implementing all the modules described herein (for e.g., modules 103, 105, 107, 203, 205, 209, 211, 223, 225, 227, 307, 407, 409, 410, 411, 419, 421, 423, 425, 427, 503, 505, 507, 509, etc.).

Referring to FIG. 8, the computer system 801 can include a bus 821 or other communication mechanism for communicating information, and a processor 803 coupled with bus 821 for processing information. In various embodiments, the computer system 821 can also include a memory 805, which can be a random-access memory (RAM) 805 or other dynamic storage device, coupled to bus 821 for determining instructions to be executed by processor 803. Memory also can be used for storing temporary variables or other intermediate information during execution of instructions to be executed by processor 803.

In various embodiments, the computer system 801 can further include a read only memory (ROM) 807 or other static storage device coupled to bus 821 for storing static information and instructions for processor 803. A storage device 809, such as a magnetic disk or optical disk, can be provided and coupled to bus 821 for storing information and instructions. In various embodiments, the computer system 801 can be coupled via bus 821 to a display 811, for displaying information to the system/computer user. An input device 813, including alphanumeric and other keys, can be coupled to bus 821 for communicating information and command selections to processor 803.

Consistent with certain implementations of the present disclosure, results can be provided by computer system 801 in response to processor 803 executing one or more sequences of one or more instructions contained in memory 805. Such instructions can be read into memory 805 from another computer-readable medium or computer-readable storage medium, such as storage device 809. Execution of the sequences of instructions contained in memory 805 can cause processor 803 to perform the modules and methods/processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 803 for execution. Such a medium can take many forms, including but not limited to, non-volatile media, volatile media, and transmission media.

In addition to computer readable medium, data can be provided as signals on transmission media included in a communications apparatus or system to provide sequences of one or more instructions to processor 803 of computer system 801 for execution. For example, a communication apparatus 815 may include a transceiver having signals indicative of instructions and data. The instructions and data are configured to cause one or more processors to implement the functions outlined in the disclosure herein. Representative examples of data communications transmission connections can include, e.g., telephone modem connections, wide area networks (WAN), local area networks (LAN), infrared data connections, NFC connections, etc.

It should be appreciated that the methodologies described herein, including flow charts, diagrams and accompanying disclosure can be implemented using computer system 801 as a standalone device or on a distributed network of shared computer processing resources such as a cloud computing network.

While the above description provides examples of one or more apparatus, methods, or systems, it will be appreciated that other apparatus, methods, or systems may be within the scope of the claims as interpreted by one of skill in the art.

1. A method of determining the likelihood of inflammatory bowel disease (IBD) in a subject, the method comprising:
determining sequencing data from a biological sample of a subject;
preprocessing the sequencing data, wherein the preprocessing includes:
i) filtering the sequencing data to remove rare features,
ii) normalizing the filtered sequencing data to remove sequencing coverage variability,
iii) batch effect reducing the filtered and normalized sequencing data, and
iv) generating engineered features from the sequencing data; and
calculating a likelihood of inflammatory bowel disease with a machine learning model trained and tested using a preprocessed initial dataset, the preprocessed sequencing data being used as inputs to the machine learning model.

2. The method of claim 1, wherein the method further comprises obtaining a biological sample of a subject.

3. The method of claim 1, wherein the step of generating engineered features from the sequencing data comprises generating any combination of the following engineered features: alpha diversity, types of bacterial interactions, a dysbiosis index, Firmicutes to Bacteroidetes ratio, gut microbiome health index (GMHI), healthy plane score, microbiome novelty score, principal component analysis score, and single sample network perturbation analysis score.

4. The method of claim 1, wherein the rare features comprise any one of:
one or more rare bacteria, and
one or more features that do not appear in a pre-determined proportion in the initial dataset.

5. The method of claim 1, wherein the rare features comprise one or more features that are present in 10% or less of samples in the initial dataset.

6. The method of claim 1, wherein the normalizing step comprises any one of:
centered-log ratio (CLR) normalization,
isometric log-ratio (ILR) normalization,
total sum scaling (TSS) transformation, and
arcsine square root transformation (ARS).

7. The method of claim 1, wherein the batch effect reducing step comprises any one of:
naive zero-centering,
an empirical Bayes method and
a negative binomial regression method.

8. The method of claim 1, wherein the sequencing data comprises 16S rRNA gene data.

9. The method of claim 1, comprising processing the sequencing data from the 16S rRNA gene into features comprising operational taxonomic units (OTUs), bacterial genera and/or bacterial species.

10. A method for training a machine learning model, comprising:
preprocessing an initial dataset of sequencing data to produce a preprocessed initial dataset, the initial dataset comprising non-overlapping first and second subsets, wherein the preprocessing includes:
a) filtering the initial dataset to remove rare features,
b) normalizing the filtered initial dataset to remove sequencing coverage variability,
c) batch effect reducing the filtered and normalized initial dataset, and
d) generating engineered features from the sequencing data; and
in one iteration of training the machine learning model, training the machine learning model using the first subset of the preprocessed initial dataset;
evaluating a performance of the trained machine learning model using the second subset of the preprocessed initial dataset; and
performing a further iteration of training the machine learning model based on whether a pre-determined level of performance is achieved.

11. The method of claim 10, wherein the step of generating engineered features from the sequencing data comprises generating any combination of the following engineered features: alpha diversity, types of bacterial interactions, a dysbiosis index, Firmicutes to Bacteroidetes ratio, gut microbiome health index (GMHI), healthy plane score, microbiome novelty score, principal component analysis score, and single sample network perturbation analysis score.

12. The method of claim 10, wherein the machine learning model comprises any one of: a random forest (RF) model, a k-nearest neighbors (KNN) model, a neural network, a logistic regression model, and a decision tree model.

13. The method of claim 10, The method of claim 1, wherein the rare features comprise any one of:
one or more rare bacteria, and
one or more features that do not appear in a pre-determined proportion in the initial dataset.

14. The method of claim 10, wherein the rare features comprise one or more features that are present in 10% or less of samples in the initial dataset.

15. The method of claim 10, wherein the normalizing step comprises any one of:
centered-log ratio (CLR) normalization,
isometric log-ratio (ILR) normalization,
total sum scaling (TSS) transformation, and
arcsine square root transformation (ARS).

16. The method of claim 10, wherein the batch effect reducing step comprises any one of:
naive zero-centering,
an empirical Bayes method and
a negative binomial regression method.

17. A system comprising one or more computers and one or more storage devices storing instructions that are operable, when executed by the one or more computers, to cause the one or more computers to perform operations of the method of claim 1.

18. A system comprising one or more computers and one or more storage devices storing instructions that are operable, when executed by the one or more computers, to cause the one or more computers to perform operations of the method of claim 10.
